(19) United States
(12) Patent Application Publication
(10) Pub. No.: US 2012/0183127 A1
(43) Pub. Date: Jul. 19, 2012

(54) PORTABLE RADIOGRAPHY IMAGING SYSTEM

(76) Inventor: Stephen Neushul, Rancho Palos Verdes, CA (US)

(21) Appl. No.: 13/304,318

(22) Filed: Nov. 24, 2011

Related U.S. Application Data
(60) Provisional application No. 61/417,391, filed on Nov. 26, 2010.

(51) Int. Cl. A61B 6/04

(52) U.S. Cl. 378/209

(57) ABSTRACT

A portable radiographic imaging system allowing multiple X-rays to be taken from various exposure positions and perspectives without having to move or reposition the patient. The imaging system may include a direct radiography system providing immediate processing and availability of the X-ray images that allows for the immediate evaluation and diagnosis. The imaging system includes a diagnostic imaging table for supporting and positioning a patient. A radiography imaging system is coupled to the imaging table and can be positioned as desired depending on the desired position and perspective of the X-Rays so as to receive the X-rays. The radiography device processes the X-ray energy into images that can be displayed in an integral monitor to allow for the immediate triage and diagnosis of the patient or product.

### CROSS REFERENCE TO RELATED APPLICATIONS

This application claims the benefit of U.S. Provisional Application No. 61/417,391 filed Nov. 26, 2011, entitled, Portable Radiographic Imaging System.

### BACKGROUND OF THE INVENTION

1. Field of the Invention

The present invention relates to radiography imaging equipment and more particularly to a radiography imaging system including an integrated imaging table.

2. Background

The use of X-ray and other radioscopic examinations are well known and commonly used in the examination and treatment of patients of all kinds. Moreover, such radioscopic examinations are further used in evaluating all kinds of non-patient items such as in non destructive testing (“NDT”), security evaluations or other radiography examinations. During such examinations, it common to have the X-rays or other radiography source taken and received from a number of different directions and locations.

As a common example, an injury to the chest may require X-ray images to be taken from the front of the chest as well as from lateral (side) perspectives. For the front or top view examinations, the patient may be placed prone on an imaging table with the X-ray source overhead and the imaging or film cassette underneath the table. For lateral view examinations, the patient's is typically rolled on a side or rotated at an angle allowing the X-ray to image from the lateral perspective. If the patient is unable to move or be moved, the X-ray and the imaging cassette must be moved to accommodate the desired image perspective.

As another example, a patient with severe but undiagnosed injuries may require a complete diagnosis and evaluation to determine the injuries, the extent of such injuries and to properly triage such patient. In many applications, including emergency room and military, the quick diagnosis and triage of a patient can mean the difference between life and death as well as efficiently utilizing the medical facility to maximize treatment capabilities. Patients with severe or unknown injuries, including potential spinal and neural injuries, are either unable to or should not be repositioned to accommodate the various necessary radiographic examinations for proper evaluation and triage. Similarly for patients with injuries that may be compounded by movement or that must be retrained. Limited positioning of the X-ray source, the imaging plate or film, the electrical connections, or even the imaging table may nonetheless require the undesirable movement or repositioning of such a patient.

What is needed is a radiographic imaging system that can be used with existing radiographic imaging devices such as X-ray apparatus that can fully support a patient, including a veterinary patient (or a product) without the need move the patient (or heavy or unwieldy product) yet allowing for multiple radiographic exposures from various perspectives and directions and further allowing for the immediate diagnosis and triage of such patient (or product). What is further needed is such a radiographic imaging system that is portable and self operating and sustaining, allowing the patient (or product) to have a radiography evaluation and then be transferred to a triage or evaluation location without the need to be moved from the supporting patient table or without the need to transfer cables, wires or reposition the patient.

### SUMMARY

The present invention provides a portable, fully self contained X-ray imaging system for the receiving and processing one or more X-rays or other radiology exposure without having to move the patient or product once placed on a support table of the system. The imaging system includes a radiography imaging device, such as a computed radiology or digital radiology device that is moveably coupled to a imaging table so that X-rays may be taken from most any perspective without the need to move or disturb the patient (or product) and advantageously allowing for the evaluation and diagnosis of the patient (or product) prior to having to move, treat or further evaluate. The present invention may further include a monitor and power supply allowing for the evaluation or diagnosis to be done while the patient (or product) remains on the imaging table, which may also be a trauma table.

the present invention provides a radiography imaging system for use with a radiographic energy exposing device comprising a support imaging table that is configured to support a patient, product or device. The imaging system also includes a radiography device that is adapted to capture radiographic energy from the radiographic exposing device and process that energy into readable images for the evaluation and diagnosis of the patient or product. The radiography imaging device is movable relative to the support table allowing for taking radiographic exposures of the patient from a plurality of directions and perspectives without the need to move the patient relative to the support table.

More specifically, an embodiment of the present invention provides a portable radiographic imaging system for use with an X-ray exposure device. The imaging system includes a support imaging table having a support surface for supporting a patient or product. The support surface is generally translucent to X-rays, allowing for the X-rays to pass through the table. The support table is portable, including wheels, so it can be moved while supporting the patient and particularly moved to and about the X-ray machine and room.

The imaging system also includes a direct radiography device, such as a computed radiography or digital radiography device, including a digital radiography plate, that is adapted to capture the energy from the X-rays being shot from the X-ray machine and for processing the received energy into useable data. This data is used to create images for evaluating and diagnosing the patient or product. The radiography device preferably includes a photostimulable surface, such as a phosphor plate, for capturing the X-ray energy transferred through the patient or product and a scanner assembly for receiving that energy.

The radiography device is moveably and rotatably coupled along the elongated axis of the imaging table and adapted to be moved relative to the support surface and elongated axis for receiving X-rays from a plurality of directions and perspectives without the need to move the patient (or product) being supported on the surface of the support table. A monitor is coupled to the support table and adapted for displaying the images of the patient and patient data. A central processing unit is also coupled to the support table. A monitor may also be mounted to a frame member of the imaging table. These devices are electronically coupled to the radiology imaging device and process the received X-ray energy into images for displaying on the monitor. A power supply may be mounted to the support table eliminating the need for wires and cables while increasing portability. The processing unit may also include a wireless data card or device providing the capability to send data from the imaging system to another location.

### BRIEF DESCRIPTION OF THE DRAWINGS

FIG. 1 shows a perspective view of an embodiment of the present invention with an imaging device positioned underneath the imaging table.

FIG. 2 shows a perspective view of an embodiment of the present invention with the imaging device positioned outside and at a generally right angle to the patient surface of the imaging table

FIG. 3A is a top view of an embodiment of the present invention showing the imaging table and a monitor.

FIG. 3B shows a rear side view of an embodiment of the present invention

FIG. 3C is a side view of an embodiment of the present invention with the imaging device positioned underneath the imaging table and showing the imaging device positioning handle and the imaging table handle.

FIG. 3D is a perspective view of an embodiment the present invention with the imaging device positioned underneath the imaging table and showing the preferred location of the power supply, controller and monitor.

FIG. 3E shows a front view of an embodiment of the present invention showing the monitor display and handle.

FIG. 4 shows a perspective view of an embodiment of the patient table with an the imaging device positioned underneath.

FIG. 5 shows a perspective view of an embodiment of the present invention with imaging device positioned away from the imaging table.

FIG. 6 shows a perspective view of an embodiment of the present invention showing the monitor display and handle.

FIG. 7 shows a perspective partially exploded view of an embodiment of the present invention with an embodiment of the radiology device removed from the imaging table.

FIG. 8 shows a perspective view of an embodiment of the present invention with the rear cover panel of the imaging table removed to show the spring support mechanism for the radiology device.

### DETAILED DESCRIPTION OF THE PREFERRED EMBODIMENTS

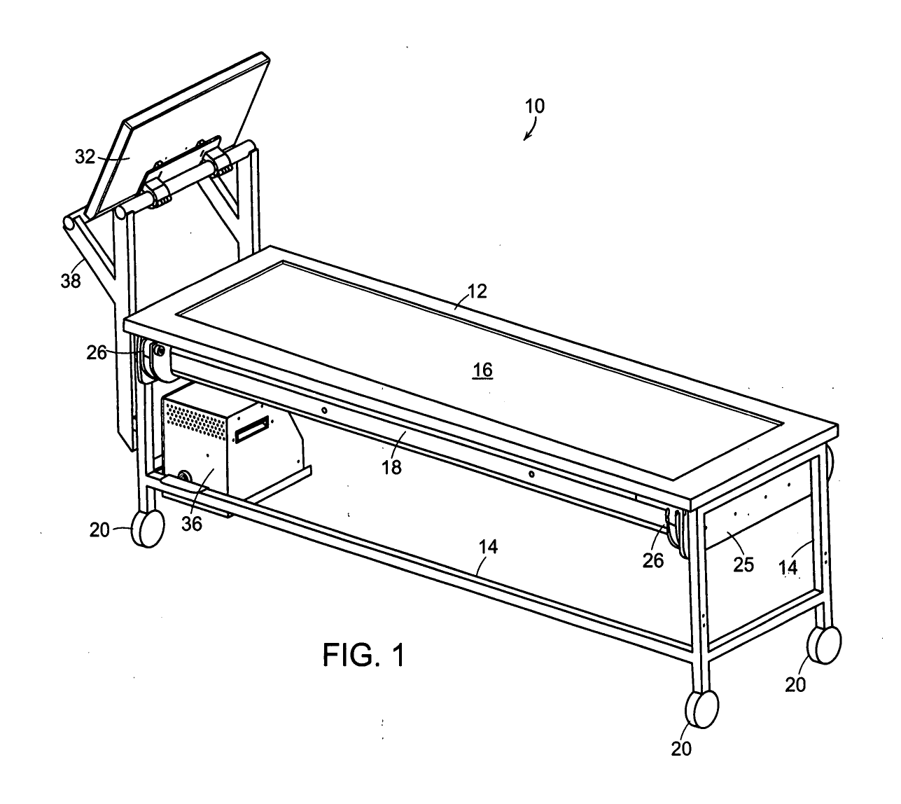

Referring now to FIGS. 1 through 5, the present invention provides a portable, fully self contained radiographic imaging system 10. Although applicable for use with various forms of radiographic energy exposures, the present invention is currently preferably adapted for receiving and processing X-rays and X-ray energy and as such, the detailed description of the embodiments that follow will identify usage with X-ray devices or combinations with an X-ray device though such disclosures are not intended to so limit the present invention.

The present imaging system 10 is adapted to allow for taking one or more X-rays of a patient, device, or product (not shown) that is placed on or against an upper support surface 12 that is part of a support table 14. The support table 14 is preferably an imaging table and may be designed as a trauma table. Preferably, upper supporting surface 12 or a substantial portion 16 thereof, is translucent to X-rays allowing them to pass through without alteration to the desired energy and wavelength. More specifically, support surface 12 may have an imaging area 16 or imaging surface that is translucent to X-rays or may comprise a filter specifically adapted to ensure a portion of the X-ray energy is blocked from passing through. Similarly, the upper support surface 12 or portion 16 may be designed to be translucent to visible light. Once placed on or against the support surface 12, the patient or product need not be moved while taking and processing multiple X-ray exposures from multiple locations, directions and even perspectives.

The imaging system 10 includes a radiography device 18 that is adapted to capture radiographic energy received from a radiographic exposing device, such as an X-ray machine (not shown) and process that energy into readable images for the evaluation and diagnosis of the patient or product. The term “patient” is intended to include all forms of patients, including veterinary patients and animals. The radiography device 18 is movably connected to the patient table 14 and adapted to be moved relative to the support surface 12 and even rotated relative to the elongated axis of the table 14, allowing for taking X-rays of the patient (or product) from a plurality of different directions and perspectives, including cross table laterals, without the need to move the patient once placed on the support surface 12.

More specifically, a preferred embodiment of the present invention provides a portable radiography imaging system 10, including an imaging table 14 for use with an X-ray exposure device. Alternatively, the present radiography exposure and imaging system 10 may include and even be coupled with an X-ray machine (not shown), such as a portable X-ray machine. The imaging system 10 includes patient support and imaging table 14 having an upper support surface 14 for supporting a patient or product (not shown). Preferably, the support surface 14 is generally flat and adapted for the medical environment but it may also be adapted for specific uses, such as pediatric, chest or spinal analysis, or simply for patient comfort. Alternatively, the imaging table may be adapted for veterinary, product evaluation, security or most any other analysis or evaluation that utilizes X-ray exposure and the reading of the resulting images. In a preferred embodiment, time support table 14 may be provided by Hokai company having a place of business in China.

Patient support surface 12 is generally translucent to X-rays, allowing for the X-rays to pass through the table surface 12. Alternatively, support surface 12 may include a section 16 that is translucent to either X-rays or visible light or both. The patient support table 14 is preferably highly portable, including wheels 20 or other rolling or sliding surfaces, so it can be moved while supporting a patient without the need to move the patient relative to the table after placement, and particularly, moved to and about the X-ray machine and the evaluations room and thereafter to the appropriate location for evaluation and treatment. As such and depending on its intended uses, the imaging table 14 may be designed to a desired length width and height and the integral radiography device 18 to a generally corresponding length. In a preferred embodiment, the imaging table 14 is a trauma table designed for both the diagnosis as well as the treatment of the patient once placed on the upper surface 12.

The radiography imaging device 18 of the present invention is preferably either a computed radiography device 18 or a direct radiography device that is adapted to receive and capture the exposure energy from the X-rays being emitted from the X-ray machine and for processing that energy into useable data. For longer imaging tables 14, a plurality or radiography devices 18 may adjoined to create a larger radiography image reading surface. The data may then be immediately used at the imaging table for evaluating and diagnosing the patient or product. The radiography device 18 preferably includes a photostimulable surface or plate, such as an imaging cassette, for capturing the X-ray energy and a scanner assembly for recovering that energy.

In a preferred embodiment, the radiography imaging device 18 or at least one of the in a multiple imaging device embodiment of the present invention, is a direct radiography device such as the iDR radiography device made by iCRco of Torrance, Calif. Radiography devices such as those described in U.S. application for patent, Ser. No. 12/197,221, entitled, “Modular Computed and Direct Radiography and Method,” filed on Aug. 22, 2008, which is hereby incorporated by reference in its entirety into this disclosure are preferred. Other radiography imaging devices contemplated also include utilizing portable direct radiography detectors, including wireless versions such as ones provided by Carestream Health (DXR-1), Canon and Fuji.

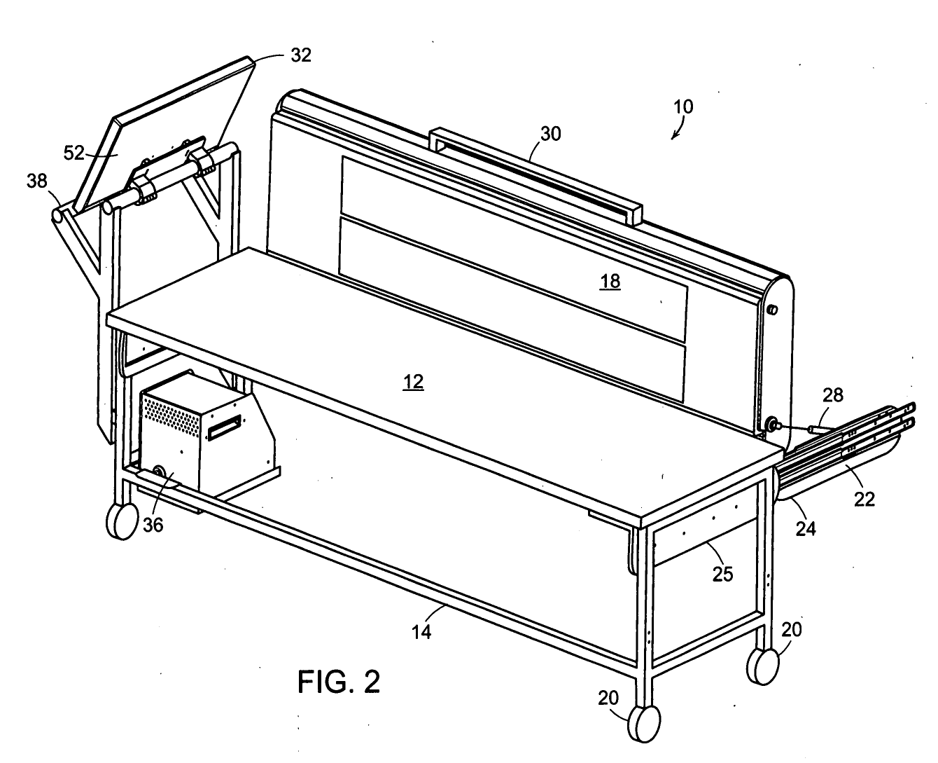

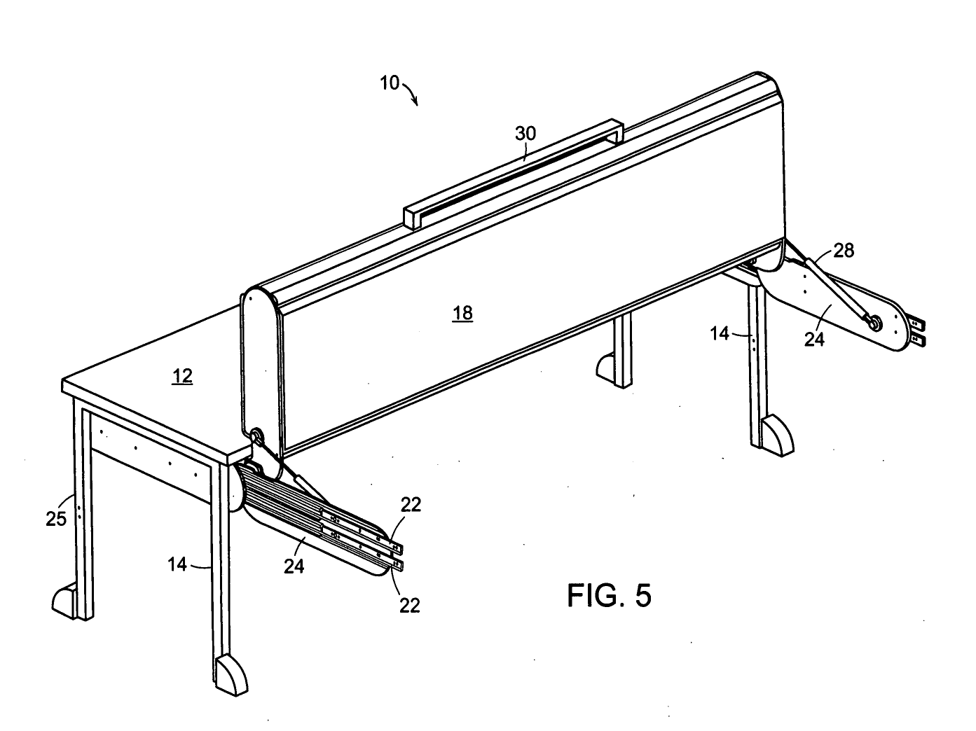

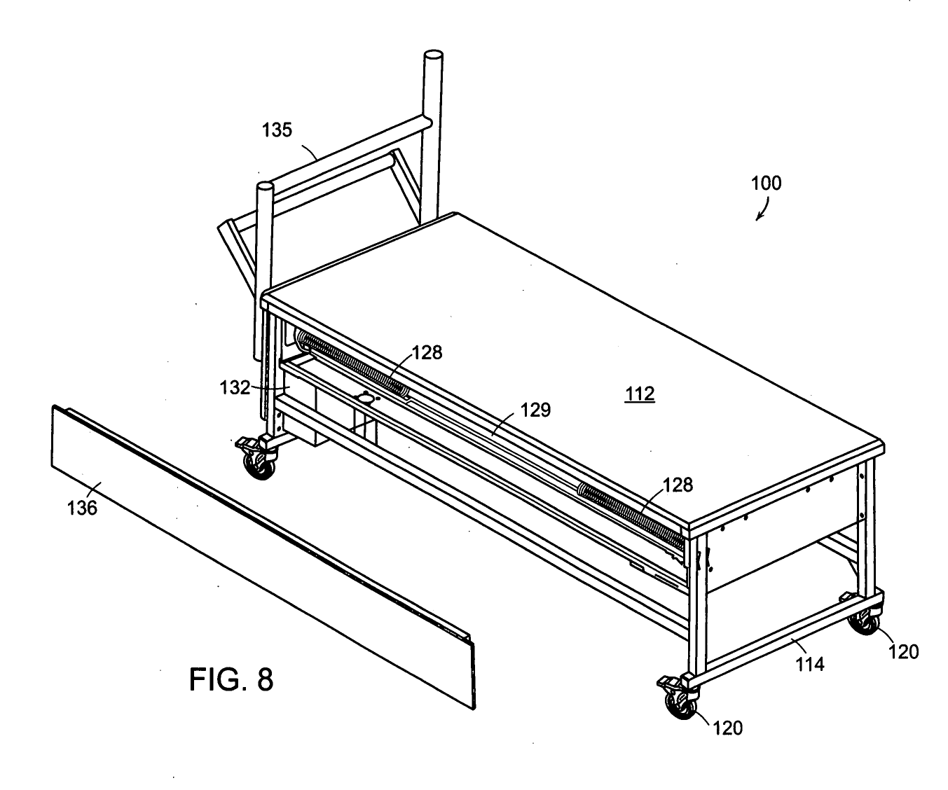

Referring now to FIG. 1 in conjunction with FIGS. 2 and 5, the radiography device 18 is moveably coupled to the support table 14 and adapted to be moved relative to the support surface 12 for receiving X-rays from a plurality of directions and perspectives without the need to move the patient once placed on the support table. In a preferred embodiment as shown, the radiology device 18 is moveably coupled to the table 14 using a plurality of slides 22, pivot plates 24 and pivot 26 (FIG. 5). A support member 28 or supporting members are coupled between the imaging table 14 and the radiography device 18 to support and provide smooth movement of the radiography device relative to the table as well as to provide and retain specific positions (ability to lock into position at various extension positions) relative to the support surface 12. In a preferred embodiment, spaced apart gas springs 28 are coupled at one end to the imaging table 14 and at the other end to the radiography device 18 and configured to generally support the weight of the radiology device 18. In a preferred embodiment, such as shown, each supporting member 28 provides between 30 to 70 pounds support or enough to counter and support the weight of the radiology device, while preferably balancing it in a plurality or positions. Additionally or alternatively, support member 28 may preferably include a coil spring assembly positioned along or even around a shaft extending along the planar axis of the radiography device 18 to similarly unweight and possibly even hold in the desired position as better shown in FIG. 8.

Alternatively, or preferably in conjunction with support member 28, a positioning system (not shown) is coupled to the table 12 and the radiology device 18 so as to allow for the positioning of the radiology device between a first position directly underneath the support surface 14 of the imaging table as shown in FIG. 1 and a second position at a generally right angle to the patient support surface (FIG. 2) and at a plurality of position there between. The positioning system may include a pivot assembly 28 (FIG. 5) on either end of the radiography device 18 that includes a position locking mechanism as is well known. Alternatively, the positioning system may include a curved track assembly on either end of the DR device 18 that further includes a locking knob assembly that can secure the radiology device at any position along the track.

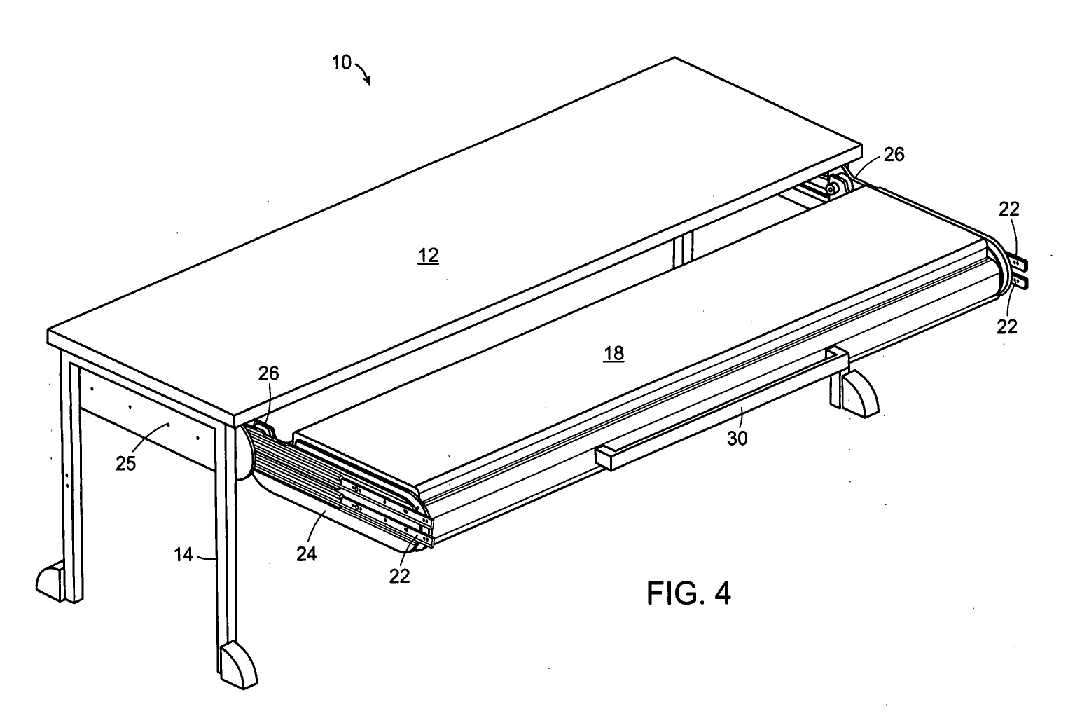

Slides 22 allow the radiology device 18 to be position directly underneath the patient support surface 12 allowing for direct and near direct overhead X-ray exposures. Slides, which may be drawer type slides, also allow the radiology device 18 to be moved outward from the table 14 by pulling on handle 30 (FIG. 4). Slides, such as drawer slides available from McMaster Car, are connected on one side to pivot plate 24 and on the other side to mounting plate 25 which is connected to table 14. Each pivot plate 24 is coupled to the DR device on either end through pivot 26 and support member 28.

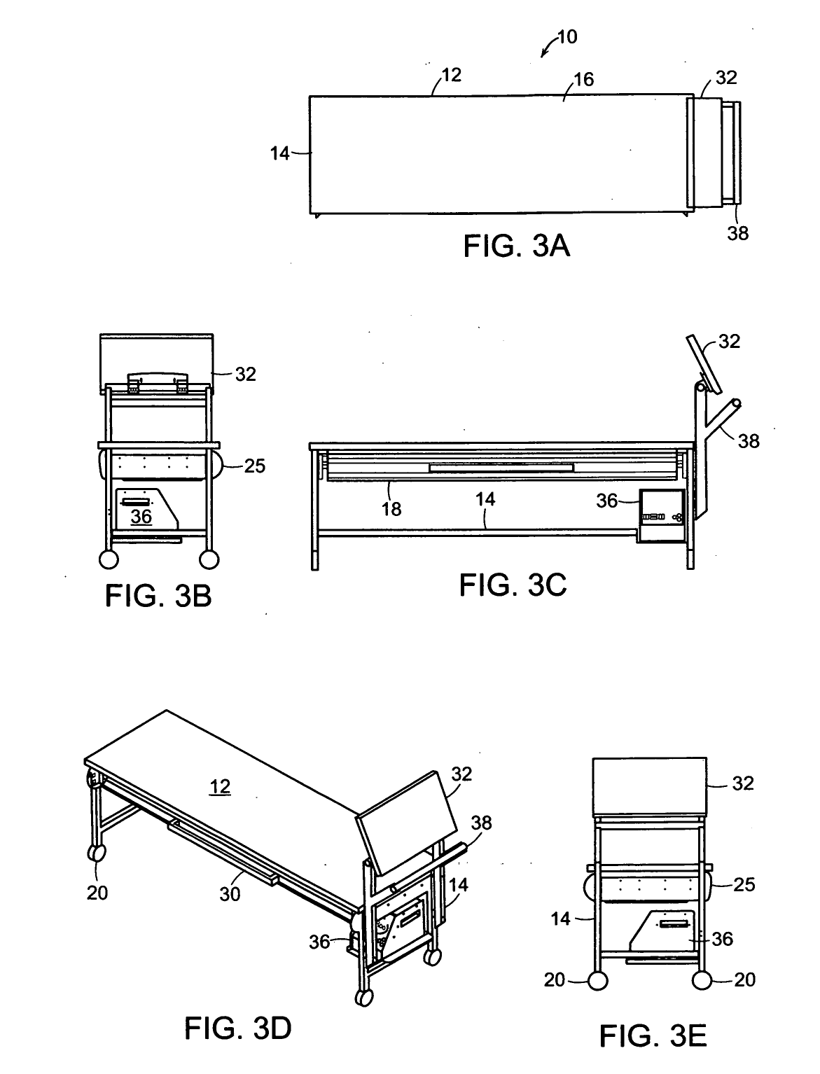

Referring now to FIG. 3 in conjunction with FIGS. 1-2 and 4-5, a central processing unit (computer) and integral monitor 32 are coupled and preferably, removeably coupled, to imaging table 14 and adapted for displaying X-ray images of the patient and patient data for immediate and convenient viewing and evaluation by a professional. In the preferred embodiment shown, the central processing unit 32 is integral with a touch screen monitor but they may also be a separate with the computer mounted to table 14 and the monitor as shown. These devices 32 are electronically coupled to the radiology device 18 and process the X-ray energy received into image data. Preferably an imaging processing software such as Clarity, Captera or even X-scan from iCRco of Torrance Calif. may be used for this purpose. Once processed, the image data may be transferred as is well known, including wirelessly.

The radiography imaging system 10 may be adapted and configured to obtain and process X-rays for particular anatomy regions or even particular animals or even products. For example piping fixtures or weldments.

In a triage or emergency room situation, the system 10 is adapted to have the patient placed on the support surface 12 and the system moved to position for X-raying. In the preferred embodiment, the DR device 18 is sufficient to receive X-rays along the entire desired length of the patient. If X-rays are desired from another angle, such as lateral views, then either the X-ray head is repositioned or the system 10 may be reposition, all with patient remaining on the table surface 12 without movement. During this repositioning, the DR device 18 is scanning the previously taken X-rays and preparing for the next exposure. Once enough X-ray images are taken, the imaging system 10 may be moved elsewhere for evaluation, diagnosis, triage, other patients or storage. There is no need to reposition the radiography device 18 or the patient.

In a preferred embodiment, the imaging system 10 allows for a full uninterrupted 14 inch by 51 inch image (of course, any size image can be provided) using a single X-ray exposure. This provides for images that do not require stitching and have no artifacts or motion blurring. The images can be modified to specific regions of concern or expanded to view the overall status. The images are made and provided right at the patient in real time, though wireless transmission of data allows such images to be sent for remote viewing and evaluation.

In a preferred highly portable embodiment, a power supply 36 may be mounted to the imaging table 14 eliminating the need for power cables, data or internet wires or cables while increasing portability of the invention. Preferably, the power unit 32 includes a power filter and may include an uninterruptible power supply and can be quickly removed and replaced. Alternatively, the system 10 may be coupled to a more traditional power supply as is well known.

The processing unit 32 may also include a wireless data card or device providing the capability to send data from the imaging system 10 to any other location capable of receiving the digital data

A handle assembly 38 is coupled to or made part of the imaging table 14 to facilitate moving the system 10. As shown, the handle assembly 38 may also be used to support patient files as well as doctors notes. The imaging table may also be specifically designed as a trauma evaluation and treatment table.

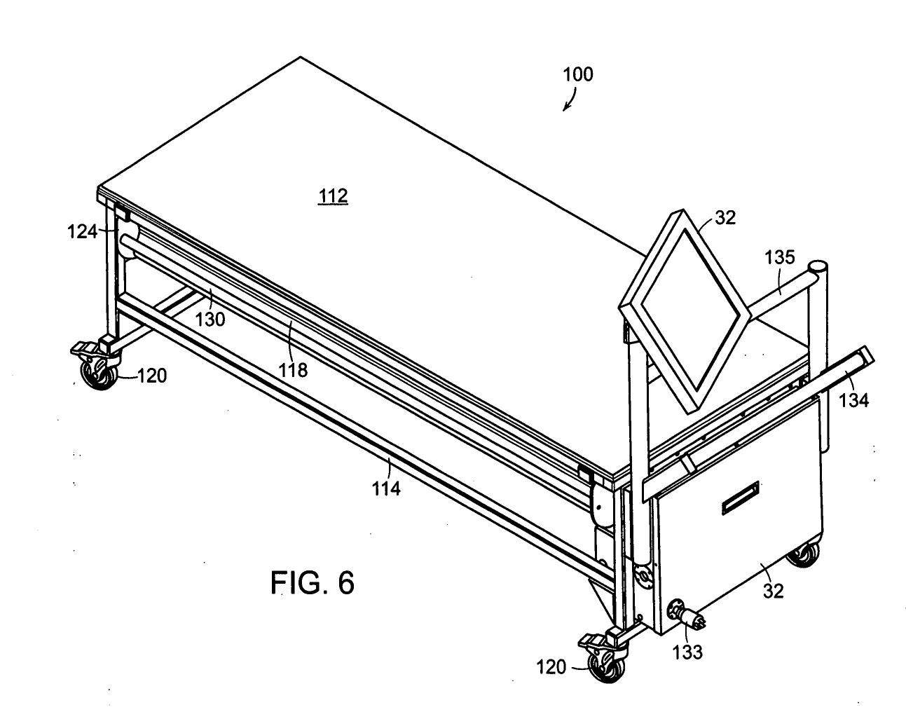

Referring now to FIGS. 6-8, another preferred embodiment of the present invention is shown. In this embodiment, a portable radiography imaging system is similarly adapted for use with an X-ray (or other radiographic energy) exposure device. As noted, the X-ray device may be a portable of fixed device and may even be directly coupled to the imaging table 114. The imaging system 100 includes patient support and imaging table 114 having an upper support surface 112 for supporting a patient or product (not shown). The imaging support surface 114 is adapted for the patients or products to be X-rayed and evaluated. This preferred embodiment of the radiography imaging system 100 is substantially similar to the previously described embodiment, however, utilizes a different support system and support members for supporting and moving the integral radiography device.

The present embodiment of the radiography imaging system 100 includes a radiography device 118 that is adapted to capture radiographic energy received from the exposing X-ray machine (not shown) and process that energy into readable images for the evaluation and diagnosis of the patient or product. A computer 132 is mounted on the imaging table 114 and preferably removable for replacement or service as needed. An electrical connection 133 is provided, and an Ethernet or other connection may similarly be provided. Connect. The imaging table may include a handle 134 or steering device as well as various support member 135 adapted to support a monitor 132 or monitors or other devices.

The radiography device 118 is supported by and moveably coupled to the support table 114 through a support housing 121. The support housing and radiography device 118 are adapted to be moved relative to the patient support surface 112 so as to be able to receive X-rays from a plurality of directions and perspectives without the need to move the patient once placed on the support table. As shown, the radiology device 118 is mounted to the support housing 121, which is itself moveably coupled to the table 114 using a plurality of slides 122, pivot plates 124 and pivot 126. The slides 122 provide for the smooth sliding movement of the radiology device relative to the support housing 121 and the imaging table 114.

A plurality of spaced apart supporting members 128 are coupled between the support housing 121 and the radiography device 118 along the elongated rear axis of the radiography device (opposite the handle 130) to support, including unweighting, and provide smooth rotational movement of the radiography device relative to support housing 121 and the imaging table 114. The support members 128 as shown are preferably coil springs placed around a shaft 129 extending along the elongated axis of the radiography device 118 from one side of the support housing to the other. A cover 136 may be coupled to the support housing to cover the support members 128.

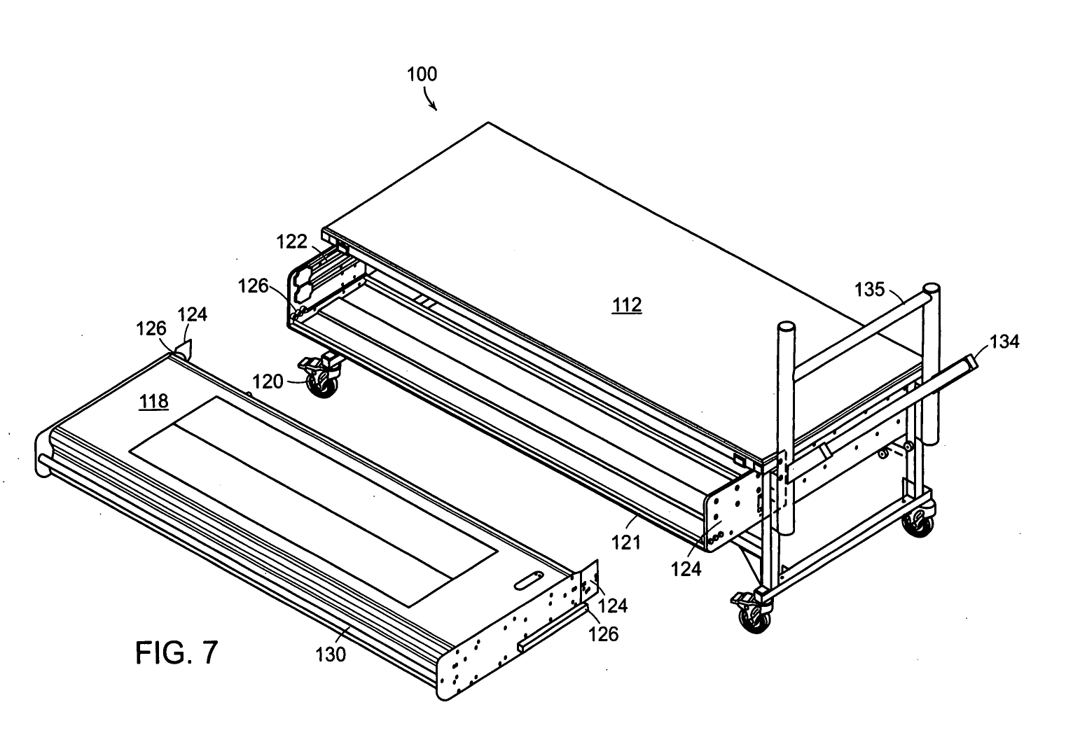

The springs 128 are preferably adapted to provide a force sufficient to generally unweight the radiography device 118 while being rotated between a first position directly underneath the support surface 112 as shown in FIG. 6 to a second position generally perpendicular to the first position and as shown in FIG. 5. The elongated shaft 129 is preferably adapted to also provide structural support to the support housing 121 and prevent any possible racking of the radiography device 118 when sliding it from underneath the imaging table 114 as shown in FIG. 6 to an extended position as shown in FIG. 7.

Similar to the other embodiments, a positioning mechanism is preferably included to secure the radiography device 118 in the desired position relative to the X-ray source and the supporting surface 112 of the imaging table 114. Bumpers (not shown) or other soft stops may also be provided to aid in preventing any slamming or hard stops of the radiology device 118. Additional support members 128, such a gas springs or shocks may also be included as described in the prior embodiment.

The embodiments of the present invention 10 and 100 advantageously allows X-rays to be taken and received along an entire body length, or a significant portion, and from various angles, positions, locations and perspectives, without the need to move the patient once placed on the table surface 12. Utilizing the preferred elongated radiography device 18, a full body may be continuously X-rayed and thereafter continuously scanned (X-ray energy detection and recovery) and resulting images evaluated. There is no need to move the radiography device 18 or detector to take full body images or to stitch multiple images together.

As noted, while the present embodiments of the invention were generally described for use with patients, and namely human patients, the imaging device may also be used or adapted for use with animals, such as in a veterinary setting, for use in non-destructive testing, for security screening, or for any type of analysis or evaluation utilizing X-rays or other radiographic energy exposure.

The foregoing description of the embodiments of the invention has been presented for the purposes of illustration and description. It is not intended to be exhaustive or to limit the invention to the precise forms or methods disclosed. Persons skilled in the relevant art can appreciate that many modifications and variations are possible in light of the above teaching.

What is claimed is:

1. A radiography imaging system for use with a radiographic energy exposing device comprising:
(a) an imaging table having an upper surface adapted to support a patient;
(b) a radiography imaging device coupled to the imaging table and adapted to capture exposure energy from the radiographic exposing device from a plurality of perspectives and process such energy into images; and
(c) wherein the radiography imaging device is movable into a plurality of positions between a first position generally under the imaging table and a second position generally lateral to the plane of the imaging table and adapted for receiving radiographic exposures from a plurality of directions and perspectives without having to move the patient relative to the imaging table.

2. The imaging system of claim 1 wherein the radiographic energy exposing device is an X-ray apparatus.

3. The imaging system of claim 2 wherein the imaging table further comprises wheels and is portable.

4. The system of claim 2 wherein the radiography imaging device comprises a direct radiography apparatus.

5. The system of claim 2 wherein the radiography imaging device comprises a computed radiography apparatus.

6. The imaging system of claim 1 further comprising a computer coupled to the imaging table, the computer adapted to process image data received from the radiography imaging device and transfer such data to a monitor.

7. The imaging system of claim 6 further comprising a monitor coupled to the imaging table

8. The imaging system of claim 6 further comprising an electrical power supply coupled to the radiography imaging device.

9. The imaging table of claim 1 comprising a patient support surface that is generally translucent to X-rays and to visible light.

10. The imaging table of claim 1 wherein the upper surface of the imaging table further comprises portions that generally block X-ray exposure energy.

11. The imaging system of claim 2 wherein the radiography imaging device comprises a scanning head assembly for scanning a phosphor based imaging plate.

12. The system of claim 2 wherein the direct radiography device comprises a portable direct radiography plate detector.

13. The imaging system of claim 1 further comprising a plurality of slides supportable coupling the radiographic imaging device to the support table and adapted to allow the imaging device to be moved relative to the support table.

14. A portable radiography system for use with an X-ray exposure device comprising:
(a) a portable imaging table having a support surface for supporting an object to be evaluated, a portion of the support surface being generally translucent to X-rays, said support table further comprising a plurality of wheels;
(b) a radiography device having an elongated X-ray exposure surface, the radiography device being adapted to receive energy emitted from the X-ray device along the exposure surface and fur processing the received X-ray energy into data for use in creating images;
(c) a positioning assembly coupled to the support table along one portion and to the radiography device along another portion, the positioning assembly adapted to allow moving the radiography device to a plurality of positions relative to the support surface for receiving X-rays from a plurality of directions; and
(d) a computer electronically coupled to the radiography device, the computer adapted for processing data received from the radiography device into images.

15. The portable imaging system of claim 14 further comprising a monitor coupled to the imaging table and electronically coupled to the computer, the monitor adapted to display images.

16. The imaging system of claim 14 further comprising a wireless device providing the capability to send data from the computer to another location.

17. The imaging system of claim 14 wherein the radiography device comprises a photostimulable surface for capturing X-ray energy and a scanning device for obtaining such energy.

18. The imaging system of claim 14 wherein the positioning assembly comprises a plurality of spaced apart slide and pivot assemblies.

19. The imaging system of claim 18 wherein a first slide and pivot assembly moveably couples an inner side of the imaging table with an external side of the radiography device and a second slide and pivot assembly moveably couples a second inner side of the imaging table with a second external side of the radiography device such that the radiography device is movable into a plurality of positions between a first position generally under the imaging table and a second position generally lateral to the plane of the imaging table.

20. The imaging system of claim 18 further comprising an elongated support member having a first end coupled to the imaging table and a second end coupled to the radiography device and adapted so support the radiography device in a plurality of positions relative to the imaging table.
